(19) United States
(12) Patent Application Publication
(10) Pub. No.: US 2024/0006040 A1
(43) Pub. Date: Jan. 4, 2024

(54) METHODS AND SYSTEMS FOR MEDICATION MANAGEMENT AND EFFECTIVENESS QUANTIFICATION

(71) Applicant: KONINKLIJKE PHILIPS N.V., EINDHOVEN (NL)

(72) Inventors: Salvatore Saporito, Rotterdam (NL); Warner Rudolph Theophile Ten Kate, Eindhoven (NL); Felipe Maia Masculo, Eindhoven (NL)

(21) Appl. No.: 18/039,731

(22) PCT Filed: Dec. 7, 2021
(86) PCT No.: PCT/EP2021/084483

§ 371 Date: Jun. 1, 2023

Related U.S. Application Data
(60) Provisional application No. 63/122,507, filed on Dec. 8, 2020.

(51) Int. Cl. G16H 20/10; G16H 40/67

(52) U.S. Cl. CPC G16H 20/10; G16H 40/67

(57) ABSTRACT

A method (100) for analyzing medication dosage for a patient (210), comprising: (i) receiving (120) a signal indicative of an activity behavior of the patient over a first time period, wherein the signal is collected by a sensor of a wearable device (220) worn by the patient; (ii) receiving (130) information about medication intake by the patient during the first time period, wherein the information comprises both medication dosage and medication intake times during the first time period; (iii) analyzing (140) the received signal and received medication intake information to determine an effectiveness of the medication on the patient, wherein the effectiveness can indicate an overdosage or underdosage of the medication during the first time period, and wherein analyzing the received signal and received medication intake information comprises generating a medication status score, the medication status score configured to minimize patient-controlled variability in the activity behavior of the patient.

### FIELD OF THE INVENTION

The present disclosure is directed generally to systems and methods for analyzing medication dosage for a patient using a monitoring system.

### BACKGROUND

Many chronic conditions require pharmacological management, such as rheumatoid arthritis, hypertension, heart failure, epilepsy, Parkinson's disease, osteoarthritis, and many more. The type of medication, as well as the dosage and frequency of intake, are typically prescribed by a physician based on patient symptoms (anamnesis) which are self-reported and/or based on functional measurements measured in a clinic setting. The medication regime is often updated periodically by care professional based on patient anamnesis, self-reported anecdotes, and/or on subjective diary information.

However, this process has several limitations. For example, physicians cannot easily adequately tailor their prescriptions to the patient. The anamnesis can be inaccurate or unclear. The measurements provide a non-representative view of a person status and medication effectiveness (e.g. awareness of test circumstances changes status, white coat hypertension, forgetfulness, bias on the specific day the questions are asked, reporting bias, and more). Further, medication regimes are typically adjusted in a trial and error fashion, often awaiting a next visit. Today prescription is based on observation while the patient is in the clinic, which is time consuming and obtrusive to the patient, being required to self-report and to present themselves at the clinic for examination, possibly staying there for a relatively long duration.

Further, overdosage or underdosage can result in negative side effects, although these can be minimized by optimizing the dosage. For example, antihypertensive medications may cause hypotension and balance issues, and diuretics may cause electrolyte imbalances. In Parkinson's patients, a too large, or a too frequent dose can lead to dyskinesia.

### SUMMARY OF THE INVENTION

Accordingly, there is a continued need in the art for methods, devices, and systems that more objectively analyze medication dosage for a patient, thereby improving detection of overdosage and underdosage.

The present disclosure is directed to inventive methods and systems for a medication dosage monitoring process. Various embodiments and implementations herein are directed to a method for analyzing medication dosage for a patient using a monitoring system. The monitoring system receives, collected by a sensor of a wearable device worn by the patient, a signal indicative of an activity behavior of the patient over a first time period. The monitoring system also receives information about medication intake by the patient during the first time period. The information comprises both medication dosage and intake time(s) during the first time period. The system analyzes the received signal and received medication intake information to determine an effectiveness of the medication on the patient, where the effect can indicate an overdosage or underdosage of the medication during a subset of the first time period. The monitoring system analyzes the received signal and the received medication intake information to create a medication status score. The medication status score is configured to minimize influence of patient-controllable variability in the activity behavior of the patient, such as patient-controlled walking power among many other possible activity behaviors.

Generally, in one aspect, a method for analyzing medication dosage for a patient is provided. The method includes: (i) receiving a signal indicative of an activity behavior of the patient over a first time period, wherein the signal is collected by a sensor of a wearable device worn by the patient; (ii) receiving information about medication intake by the patient during the first time period, wherein the information comprises both medication dosage and medication intake times during the first time period; and (iii) analyzing the received signal and received medication intake information to determine an effectiveness of the medication on the patient, wherein the effectiveness can indicate an overdosage or underdosage of the medication during the first time period, and wherein analyzing the received signal and received medication intake information comprises generating a medication status score, the medication status score configured to minimize patient-controlled variability in the activity behavior of the patient.

According to an embodiment, the medication status score is generated by the steps of: (i) identifying one or more events in the received signal based on selection criteria; (ii) extracting a quality metric from each of the identified one or more events; and (iii) aggregating the extracted quality metrics from the one or more events for the first time period.

According to an embodiment, the extracted quality metric comprises walking intensity, heart rate variability, chair rise peak power during a sit-to-stand transition, or a combination thereof.

According to an embodiment, the received signal and received medication intake information are received remotely from the patient.

According to an embodiment, the sensor is an accelerometer, a gyroscope, a magnetometer, a photopletysmograph, galvanic skin response, an air pressure sensor, a thermometer, an SpO₂ sensor, an ECG sensor, or a combination thereof.

According to an embodiment, the medication status score is displayed on a user interface.

According to an embodiment, the display comprises a time series visualization for some or all of the first time period.

According to an embodiment, the method further includes the step of receiving an adjustment, in response to an indication of an overdosage or underdosage of the medication during a subset of the first time period, of a dosage of the medication.

According to an embodiment, the method further includes the step of receiving, from a physician monitoring the patient, an annotation for a time point during the first time period.

According to a second aspect is a system configured to analyze medication dosage for a patient. The system includes: (i) a communication module configured to receive: (1) a signal indicative of an activity behavior of the patient over a first time period, wherein the signal is collected by a sensor of a wearable device worn by the patient; and (2) information about medication intake by the patient during the first time period, wherein the information comprises both medication dosage and intake times during the first time period; (ii) a processor configured to analyze the received signal and received medication intake information to determine an effectiveness of the medication on the patient, wherein the effect can indicate an overdosage or underdosage of the medication during a subset of the first time period, and wherein analyzing the received signal and received medication intake information comprises generating a medication status score, the medication status score configured to minimize patient-controlled variability in the activity behavior of the patient; and (iii) a display configured to display one or more of the received signal, the medication intake information, and/or the medication status score over the first time period.

According to an embodiment, the processor is further configured to generate the medication status score by: (i) identifying one or more events in the received signal based on selection criteria; (ii) extracting the identified one or more events; and (iii) aggregating the extracted events for the first time period.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program” or “computer program” are used herein in a generic sense to refer to any type of computer code (e.g., software or microcode) that can be employed to program one or more processors or controllers.

In one network implementation, one or more devices coupled to a network may serve as a controller for one or more other devices coupled to the network (e.g., in a master/slave relationship). In another implementation, a networked environment may include one or more dedicated controllers that are configured to control one or more of the devices coupled to the network. Generally, multiple devices coupled to the network each may have access to data that is present on the communications medium or media; however, a given device may be “addressable” in that it is configured to selectively exchange data with (i.e., receive data from and/or transmit data to) the network, based, for example, on one or more particular identifiers (e.g., “addresses”) assigned to it.

The term “network” as used herein refers to any interconnection of two or more devices (including controllers or processors) that facilitates the transport of information (e.g. for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for analyzing medication dosage for a patient, in accordance with an embodiment.

FIG. 2 is a schematic representation of a system configured to analyze medication dosage for a patient, in accordance with an embodiment.

FIG. 3 is a schematic representation of a wearable device in accordance with an embodiment.

FIG. 4 is a graph of six weeks of sensor data from a Parkinson patient, in accordance with an embodiment.

FIG. 5A is a graph of more than 100 weeks of data from a Parkinson patient, in accordance with an embodiment.

FIG. 5B is a graph of more than 100 weeks of data from a Parkinson patient, in accordance with an embodiment.

FIG. 6 is a graph of walking data as detected by an accelerometer of a wearable device, in accordance with an embodiment.

FIG. 7 is a graph quality metric extracted from the received sensor data, in accordance with an embodiment.

FIG. 8 is a histogram of a walk intensity quality metric generated in five second windows, in accordance with an embodiment.

FIG. 9A is a graph of gait quality correction based on effort level, in accordance with an embodiment.

FIG. 9B is a graph of gait quality correction based on effort level, in accordance with an embodiment.

FIG. 10 is a graph of event selection in the 120 minutes before and after medication intake, in accordance with an embodiment.

FIG. 11A is a graph of event selection, in accordance with an embodiment.

FIG. 11B is a graph of event selection, in accordance with an embodiment.

FIG. 11C is a graph comparing walk quality before and after medication intake without selection for effort level, in accordance with an embodiment.

FIG. 11D is a graph comparing walk quality before and after medication intake with selection of effort level, in accordance with an embodiment.

FIG. 12 is a graph showing aggregation of selected events from multiple days, in accordance with an embodiment.

FIG. 13 is a graph of daily walking intensity changes over a period of six weeks, in accordance with an embodiment.

FIG. 14 is a flowchart of a method for analyzing medication dosage for a patient, in accordance with an embodiment.

### DETAILED DESCRIPTION OF EMBODIMENTS

The present disclosure describes various embodiments of a system configured to analyze medication dosage for a patient. More generally, Applicant has recognized that it would be beneficial to provide a medication dosage system that more objectively analyze medication dosage for a patient, thereby improving detection of overdosage and underdosage. In view of the foregoing, various embodiments and implementations are directed to a method for analyzing medication dosage for a patient. A signal indicative of an activity behavior of the patient over a first time period is received, the signal being collected by a sensor of a wearable device worn by the patient. Information about medication intake by the patient during the first time period is received, the information including both medication dosage and intake time(s) during the first time period. The received signal and received medication intake information are analyzed to determine an effectiveness of the medication on the patient, where the effect can indicate an overdosage or underdosage of the medication during a subset of the first time period. The analysis includes generation of a medication status score configured to minimize influence of patient-controllable variability in the activity behavior of the patient.

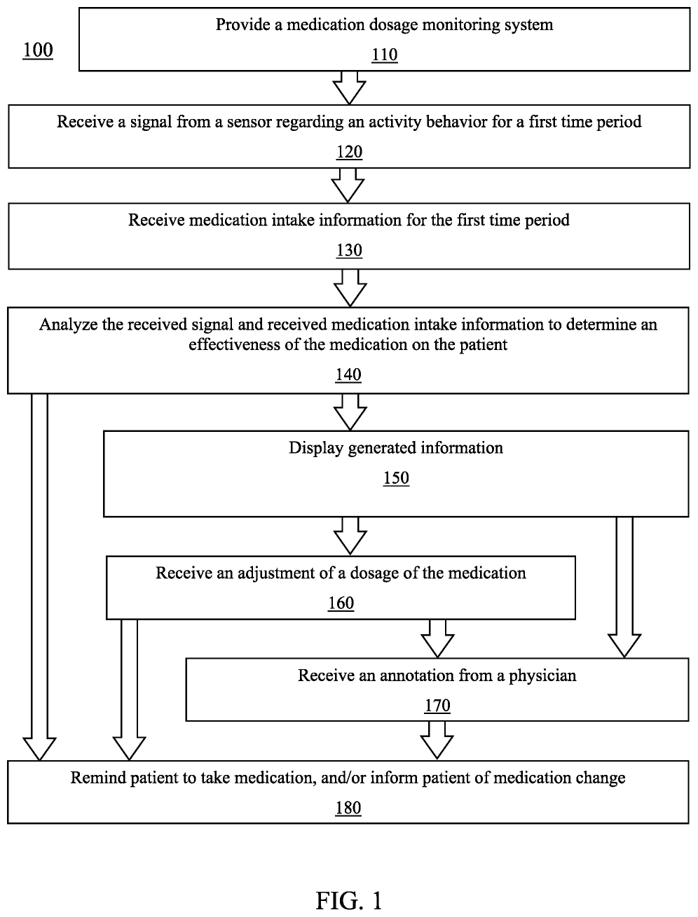

Referring to FIG. 1, in one embodiment, is a method 100 for analyzing medication dosage for a patient using a monitoring system. At step 110 of the method, a monitoring system is provided. The monitoring system can be any of the systems described or otherwise envisioned herein. As described or otherwise envisioned herein, the monitoring system is a wearable device, a remote server, or any other system.

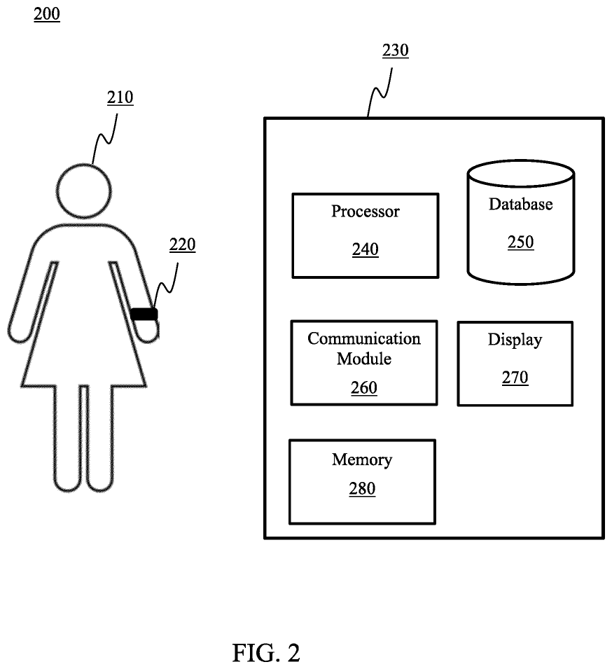

Referring to FIG. 2, in accordance with an embodiment, is a schematic representation of a medication dosage monitoring system 200. Although shown as multiple different components, this is a non-limiting embodiment of the system. A single device or component may comprise the entire system, or the system may comprise multiple components.

According to an embodiment, medication dosage monitoring system 200 comprises a wearable device 220 worn by a patient or user 210. The patient or user 210 is experiencing or believed to be experiencing a chronic disease that requires medication. Although wearable device 220 is shown on the wrist, the wearable device may be worn, carried, implanted, or otherwise adhered or in communication in any way with the patient or user 210.

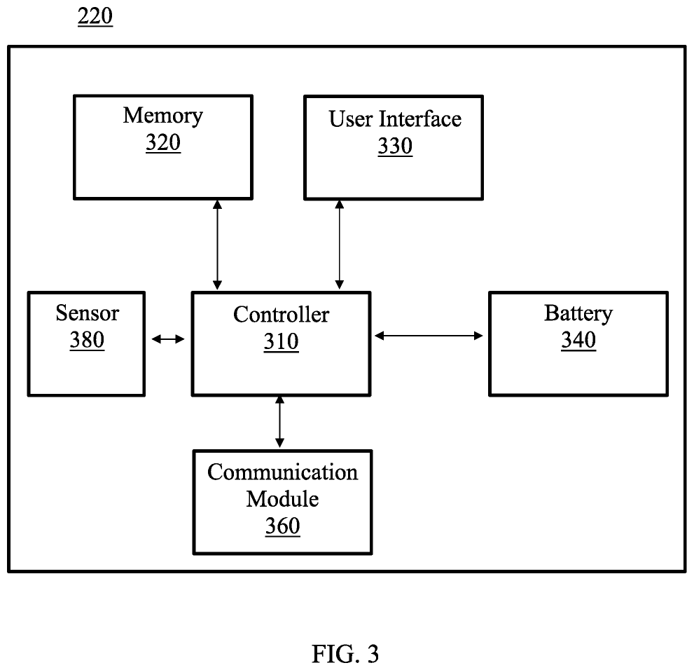

Referring to FIG. 3 is an embodiment of wearable device 220 worn by a patient or user 210. The device 220 comprises a controller 310 that controls or organizes one or more functions of the device. The controller 310 is capable of executing instructions stored in memory 320 or other data storage or otherwise processing data to, for example, perform one or more steps of the method. Controller 310 may be formed of one or multiple modules. Controller 310 may take any suitable form, including but not limited to a microprocessor, microcontroller, multiple microcontrollers, circuitry, field programmable gate array (FPGA), application-specific integrated circuit (ASIC), a single processor, or plural processors.

The device further comprises a memory 320 to store data such as sensor data, user settings, and/or other information. Memory 320 can take any suitable form, including a non-volatile memory and/or RAM. The memory 320 may include various memories such as, for example L1, L2, or L3 cache or system memory. As such, the memory 320 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of the device. It will be apparent that, in embodiments where the processor implements one or more of the functions described herein in hardware, the software described as corresponding to such functionality in other embodiments may be omitted.

Wearable device 220 further includes a batter 340 to power the device, and a user interface 330 to receive input from the user and/or to provide output to the user. User interface 330 may comprise an input/output device, a haptic device, a touch screen, an optical display, a microphone, a keypad, a keyboard, a pointing device, an image capture device, a video camera, an audio output device, or any combination thereof.

A communication module 360, which can be wired or wireless, facilitates communication with one or more other components of the medication dosage monitoring system 200. Wearable device 220 comprises a sensor 380 configured to obtain sensor data related to an activity behavior of the user. The activity behavior may be any activity that can be utilized to inform medication dosage. For example, the activity behavior may be walking or other motion by the patient, heart rate variability, chair rise peak power during a sit-to-stand transition, or any other activity. The sensor may be any sensor configured to or capable of obtaining the sensor data utilized in the methods and systems described or otherwise envisioned herein. For example, the sensor may be any of an accelerometer, a gyroscope, a magnetometer, a photopletysmograph, galvanic skin response sensor, and a combination thereof, among other possible sensors.

Returning to FIG. 2, the medication dosage monitoring system 200 comprises a medication dosage analysis component 230, which receives the sensor information from the wearable device 220 via a wired and/or wireless communication module 260. The medication dosage analysis component 230 may be remote from the wearable device 220 and thus the patient 210. For example, the wearable device may communicate the sensor information over a wired and/or wireless network such as the internet, an intranet, or any other network that facilitates remote communication.

The medication dosage analysis component 230 further comprises a processor or controller 240 that facilitates or controls one or more functions of the component. The controller 240 is capable of executing instructions stored in memory 280 or other data storage or otherwise processing data to, for example, perform one or more steps of the method. Controller 240 may be formed of one or multiple modules. Controller 240 may take any suitable form, including but not limited to a microprocessor, microcontroller, multiple microcontrollers, circuitry, field programmable gate array (FPGA), application-specific integrated circuit (ASIC), a single processor, or plural processors.

The component further includes a database 250 to store information such as the received sensor information. Since the component is configured to receive information about medication intake by the patient during a time period, comprising both medication dosage and intake time(s), the database 250 can further store or otherwise comprise this information. The medication intake information can come from the patient's prescription, a diary or other report from the patient, and/or from other sources.

The medication dosage analysis component 230 further comprises a display 270 which is configured to receive input from a user of the component, and/or to provide output to the user of the component. Display 270 may comprise an input/output device, a haptic device, a touch screen, an optical display, a microphone, a keypad, a keyboard, a pointing device, an image capture device, a video camera, an audio output device, or any combination thereof. According to one embodiment, display 270 is configured to provide an analysis of the received signal and received medication intake information to a physician or other healthcare professional. For example, the analysis can be utilized to determine an effectiveness of the medication on the patient, wherein the effectiveness can indicate an overdosage or underdosage of the medication during a subset of the first time period.

While medication dosage analysis system 200 is shown as including one of each described component, the various components may be duplicated in various embodiments. For example, a controller may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, a controller may include a first processor in a first server and a second processor in a second server. Many other variations and configurations are possible.

Returning to method 100 in FIG. 1, at step 120 of the method the system receives, via a wired and/or wireless communications connection or network, sensor information from wearable device 220. The sensor information is obtained for and indicative of an activity behavior of the user. The activity behavior may be any activity that can be utilized to inform medication dosage. For example, the activity behavior may be walking or other motion by the patient, heart rate variability, chair rise peak power during a sit-to-stand transition, or any other activity. The sensor may be any sensor configured to or capable of obtaining the sensor data utilized in the methods and systems described or otherwise envisioned herein. For example, the sensor may be any of an accelerometer, a gyroscope, a magnetometer, a photopletysmograph, galvanic skin response sensor, an air pressure sensor, thermometer, SpO₂ sensor, ECG sensor, and a combination thereof, among many other possible sensors.

According to an embodiment, continuous monitoring of a patient's activity outside of a clinical setting has the potential to objectively assess the effect of different doses and medication timings on subject status, enabling optimization of the therapeutic window and to reduce side effects. As an example, walking difficulties for Parkinson's patients are managed pharmacologically using levodopa-based medications. An overdosage can lead to dyskinesia, a severe side effect. According to an embodiment, the effect of medication is measurable in a controlled condition (e.g. in the clinic while same shoes, same motivation level, etc.). However, outside the clinical setting there is substantial variability in activities and volunteer effort level that hampers the detection of medication effect.

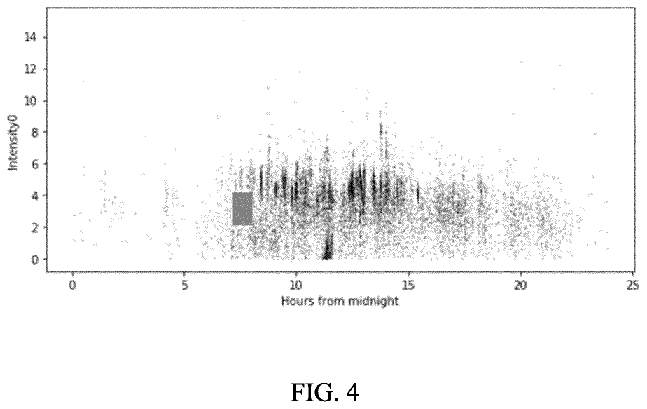

Referring to FIG. 4 is a graph of six weeks of data from a Parkinson patient, specifically sensor-based walking intensity estimated in a patient outside a clinical setting using a pendant in inverted pendulum mode. The gray bar shows the expected order of magnitude of the change induced by levodopa medication, as validated in camera controlled sessions.

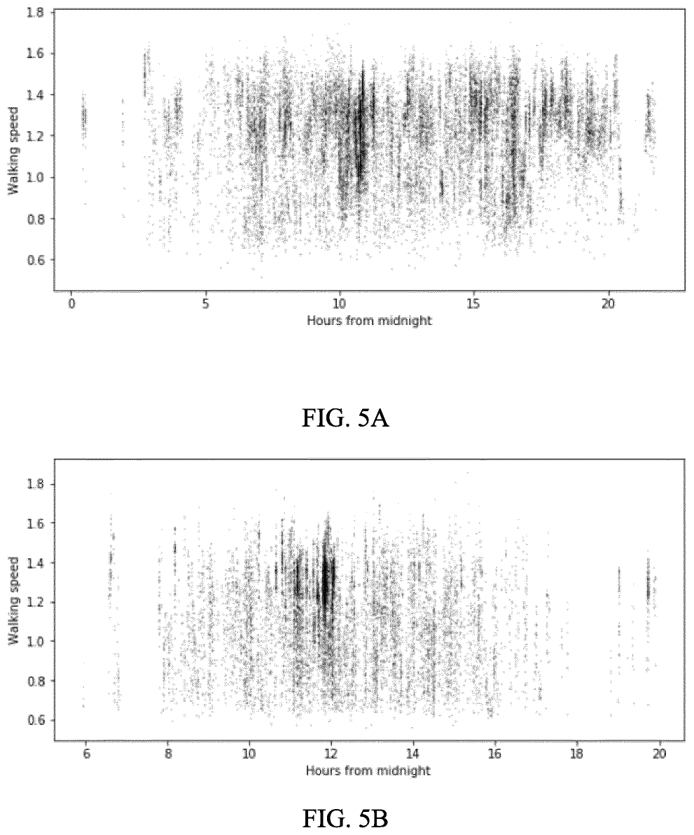

In healthy individuals, walking speed fluctuates during the day as result of subject activities and voluntary control, while their health status can be considered constant. Accordingly, referring to FIGS. 5A and 5B are graphs of more than 100 weeks of data from a Parkinson patient, specifically sensor-based walking speed estimated in a patient outside a clinical setting using a pendant in inverted pendulum mode. Walking speed seems intrinsically variable in the clinical setting, as a response to behavioral and environmental requirements that a subject experiences.

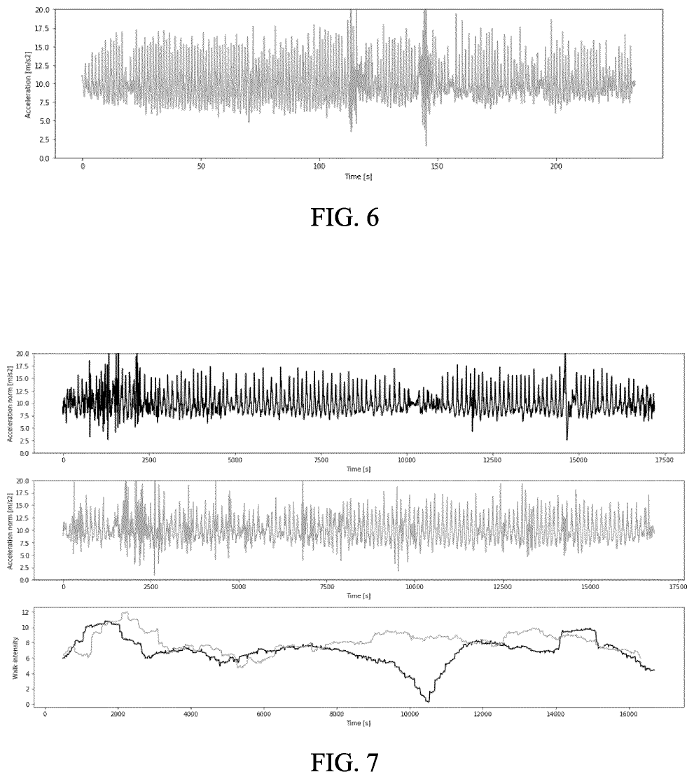

According to an embodiment, patterns from multiple days of obtained sensor data can be combined to provide an average or other form of estimate to obtain a more generic pattern for the user/patient. Referring to FIG. 6, for example, is an example of a monitored activity behavior. Specifically, the graph depicts walking data as detected by an accelerometer of a wearable device.

Returning to method 100 in FIG. 1, at step 120 of the method the system receives, via a wired and/or wireless communications connection or network, information about medication intake by the patient during some or all of the time period for which sensor data was obtained from the wearable device. According to an embodiment, the medication intake information comprises both medication dosage and intake times during the time period. The medication intake information can come from the patient's prescription, a diary or other report from the patient, filled automatically based on medication dispenser logs, and/or from other sources. TABLE 1 comprises an example of patient medication intake information obtained by one of these methods.

TABLE 1
Patient medication intake diary

| Medication | Dose | Prescribed intake time | Actual intake time |
|---|---|---|---|
| Sinemet | 150 mg | Monday 11 May | Monday 11 May |
| | | h 9.00 | h 9.12 |
| Humira | 200 mg | Monday 11 May | skipped |
| | | h 13.00 | |

The received medication intake information may be utilized by the system immediately, and/or may be stored for future analysis.

At step 140 of the method, system 200 analyzes the received signal and the received medication intake information to determine an effectiveness of the medication on the patient. According to an embodiment, the determined effectiveness can indicate an overdosage or underdosage of the medication during a subset of the first time period. According to an embodiment, analyzing the received signal and received medication intake information comprises generating a medication status score or metric, where the medication status score or metric is configured to minimize influence of patient-controllable variability in the activity behavior of the patient. The system can analyze the received information in real-time or can analyze stored information.

According to an embodiment, a quality metric is generated from the received activity behavior sensed by the sensor data. For example, activities such as walking episodes and chair rises can be detected, and a quality metric such as walking speed and maximum power exercised during a sit to stand can be generated.

The system can generate a sensor-enabled medication status score which minimizes the influence of specific activities—such as variability in the sensor data due to a freely-chosen effort level, for example relaxed walk vs. brisk walk—on the variability due to acute changes in maximum capacity resulting from medication, such as a decrease in maximum/comfortable walk speed after levodopa-based medication intake, as just one example.

According to an embodiment, the physician is provided with information generated by the system, such as patient information, the sensor data, the medication intake information, the medication status score, and/or any other information. For example, the display may comprise a graph over time of the scores, together with the pill dose and intake moments. In this way, it is not required the patient is to present himself for a day at the clinic, while the physician receives more detailed and patient specific information.

According to an embodiment, the system can be used to verify that medication intake happens and/or happens at the right time. Possibly, the verification is windowed to the moments the pill dispenser is used. Thus, the system can further be used to remind the patient to take a medication if the system detects from the generated information that a dose is missing and/or necessary.

Referring to FIG. 7, in one embodiment, a quality metric is extracted from the received sensor data. A wearable device comprising an accelerometer was worn on the wrist of a Parkinson's patient, and sensor data was collected in the home environment during unscripted, undirected activities of daily living with typical sources of variability such as different footwear, different device-wearing positions, and other variability. The top panel comprises an example of sensor data showing the user's walking during the period before levodopa-medication. The middle panel comprises an example of sensor data showing the user's walking during the period after levodopa-medication. The bottom panel comprises a corresponding log(signal variance) computed in sliding windows of five seconds. Black is before the medication, gray is after the medication. Although the quality metric is calculated as described, many other methods of calculating the quality metric are possible.

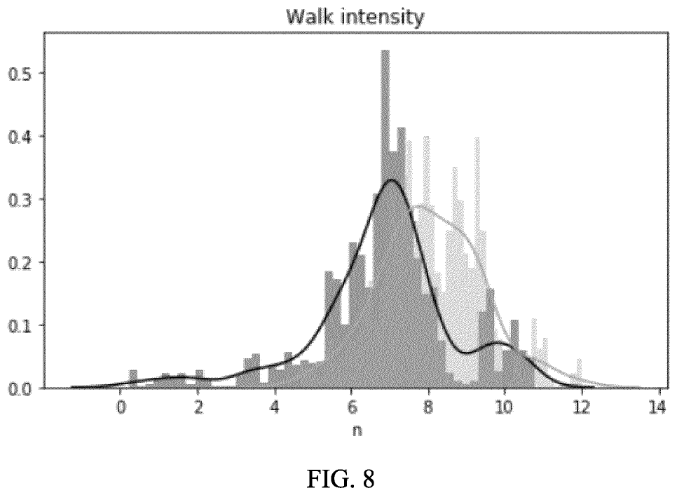

Referring to FIG. 8, in one embodiment, is a histogram of a walk intensity quality metric generated in five second windows, where darker gray is before medication and lighter gray is after medication. As expected, medication has significant effect on gait quality. Further analysis showed a significant effect over a population of 25 subjects, although that data is not shown here. Although the difference in intensity is significant, the variability in performance during a specific event hampers the detection of such effect, such as change in intensity during a walk.

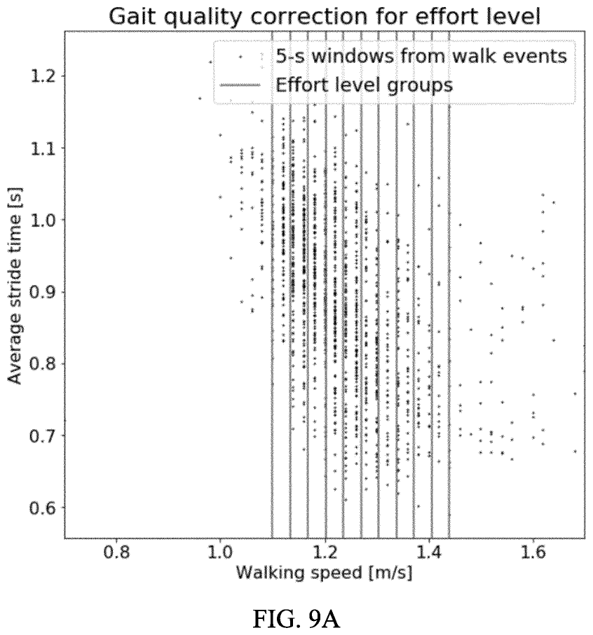

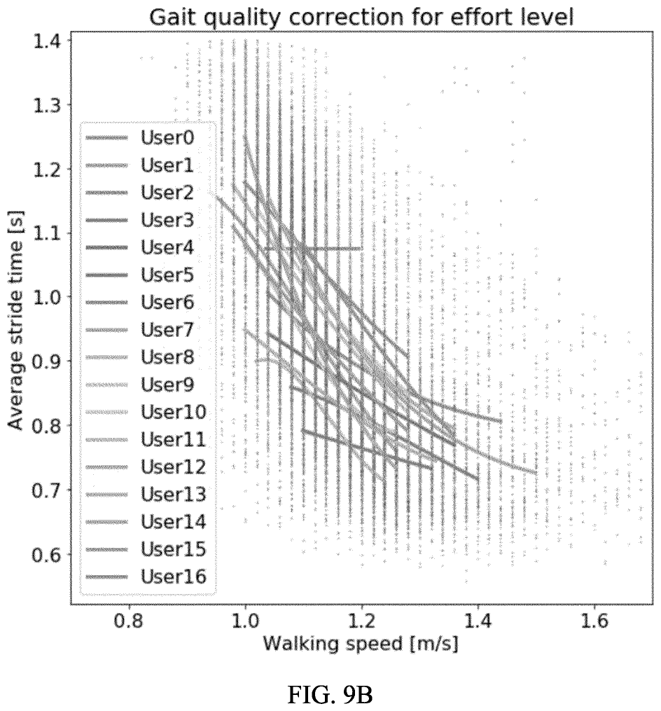

According to an embodiment, one or more events are selected from the analyzed data, based on effort level. Events corresponding to the same intensity effort can be identified and/or aggregated. For example, often but not always, high effort levels are selected as medication is expected to impact functional capacity and thus peak/quasi-peak subject performance. Referring to FIG. 9A, in one embodiment, is data from a user where a relationship is visible between effort level quantified by walking speed and gait quality, which here is stride time. Referring to FIG. 9B for the same users, the relationship between effort and quality can be estimated in a subject-specific way (shown by the solid lines), and corresponding groups can be determined on the actual effort levels of a specific user.

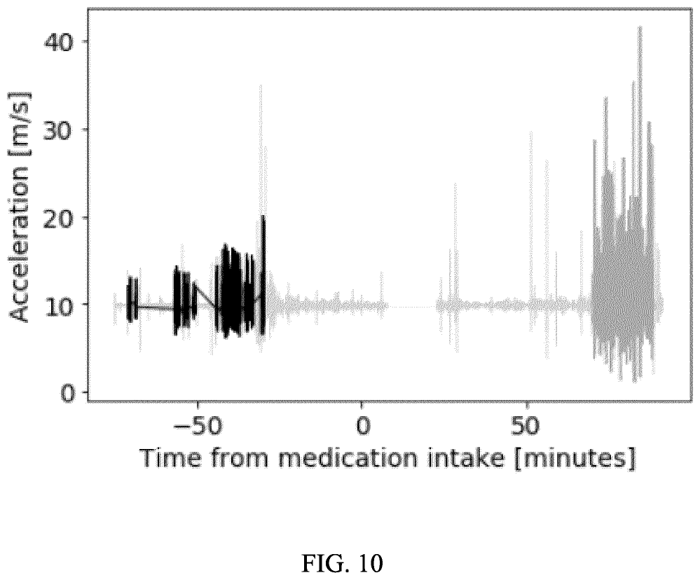

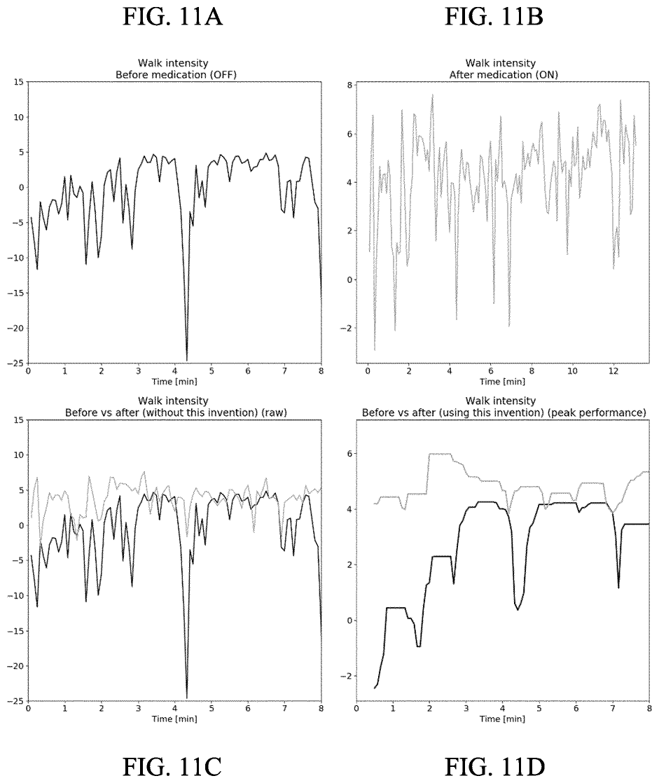

According to an embodiment, events occurring in a specific time window can be defined or informed by medication intake. For example, referring to FIG. 10 is a graph showing an example of event selection in the 120 minutes before and after medication intake. Referring to FIGS. 11A through 11B is additional event selection. FIG. 11A is a graph of walk quality indicators within the two hours before medication intake, while FIG. 11B is a graph of walk quality indicators within the two hours after medication intake, for the same individuals. FIG. 11C is a graph of the comparison of walk quality before and after medication intake without selection for effort level, and FIG. 11D is a graph of the comparison of walk quality before and after medication intake with selection of effort level, which in this graph is peak intensity per minute. Notably, intra-subject variability is large compared to the magnitude of the changes introduced by medication, and when considering the peak quality measure the difference is rendered more visible.

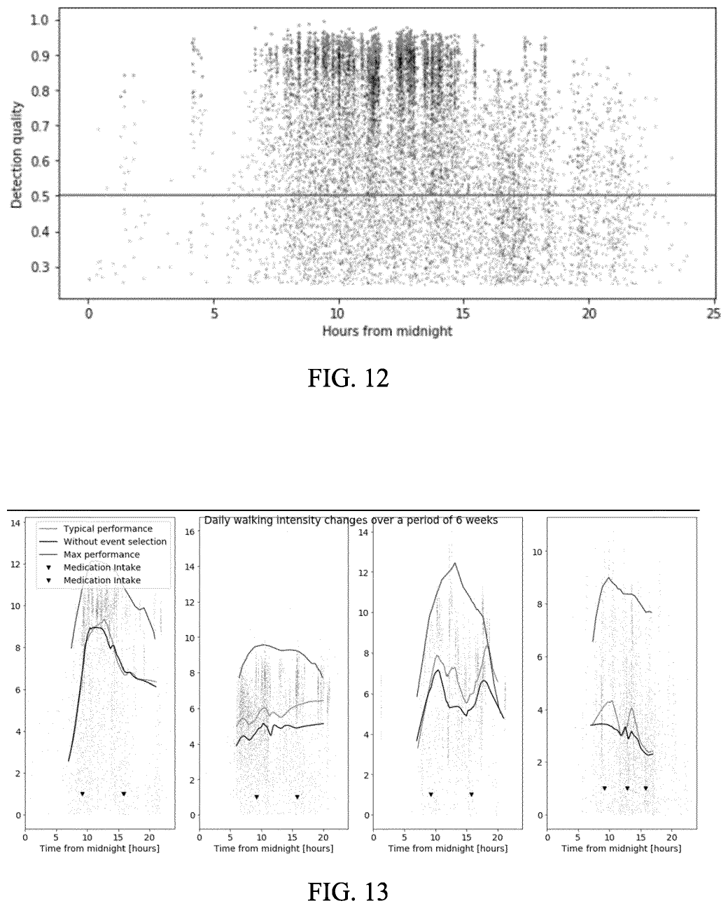

According to an embodiment data from the generated quality metric, selected for effort level, can be aggregated over a certain time period, typically on the order of days, weeks, and months. Groupings of selected events can also be aggregated based on their detection quality. Further, based on the groupings, statistical measures can be extracted from the group of events. For example, a maximum and mean can be extracted from the aggregation. Referring to FIG. 12 is an example aggregation of events from multiple days. The black points indicate all events, the red points indicate the top 10% of events with the highest quality per day, and the line represents the absolute minimum quality threshold for rejection. Other criteria for grouping events are possible, including but not limited to timing before and/or after last medication intake.

Many activity behavior metrics can be extracted from or otherwise identified in the sensor data. For example, quality metrics related to subject performances such as walking speed during a walk episode and/or heart rate variability from a photoplethysmograph may be extracted. As another example, quality metrics related to specific subject performances such as chair rise peak power during a sit to walk transition may be extracted. As another example, quality metrics related to signal quality itself such as signal to noise, presence of offset, baseline drift, and other signal quality parameters may be extracted.

There are many possible event selection criteria that may be utilized. For example, the system may select events that occur at a specific time of the day or timing related to medication intake. As another example, the system may select events having certain characteristics such as specific minimum duration and/or other characteristics. As another example, the system may select events occurring in a certain context provided by localization techniques, such as using GPS or Bluetooth location information. As another example, the system may select events occurring in certain context provided by other sensor data, such as photoplethysmography data after a specific movement as detected by accelerometer data. The event selection criteria may also be any combination of these enumerated criteria, or other criteria envisioned by this disclosure.

Returning to method 100 in FIG. 1, at step 150 the medication dosage monitoring system 200 displays some or all of the information. For example, the system may display information to a physician for analysis and dosage evaluation. According to an embodiment, the physician is provided with information generated by the system, such as patient information, the sensor data, the medication intake information, the medication status score, and/or any other information. For example, the display may comprise a graph over time of the scores, together with the pill dose and intake moments. According to one embodiment, display 270 is configured to provide an analysis of the received signal and received medication intake information to a physician or other healthcare professional. For example, the analysis can be utilized to determine an effectiveness of the medication on the patient, wherein the effectiveness can indicate an overdosage or underdosage of the medication during a subset of the first time period.

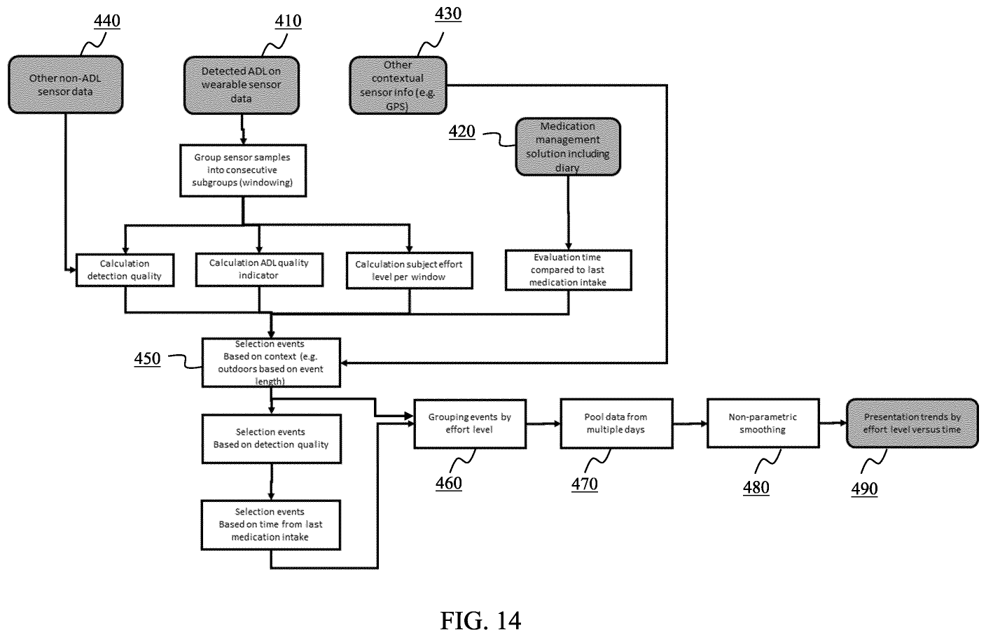

According to an embodiment, the display comprises a time series visualization presenting multiple statistical measures for an indicator, and/or a time series presenting indicators from different periods of time. Referring to FIG. 13, in one embodiment, is an example of data that may be displayed, showing daily walking intensity changes over a period of six weeks. Thus, the data comprises a sensor-based score (i.e., a walk quality indicator, namely walking intensity) for four different users, and points represent sensor scores from individual walk events during the six weeks period versus times of the day. The different lines represent non-parametric regression over the sensor-based scores of the individual events, regardless of the specific day. It is shown that aggregating on different effort level yields different patterns for the sensor score. An end-user could visualize this information jointly with medication intake. As just an example, the triangular markers in the figures demonstrate medication intake timing each date.

At optional step 160 of the method, the system may receive an instruction to adjust a dosage of the medication taken by the patient. For example, a healthcare professional reviewing the displayed information may determine that there is an underdosage or an overdosage and thus that the amount of medication at an intake or over a day needs adjustment, that the timing of medication intake needs adjustment, or another changes needs to be made to the medication dosage as a result of the information displayed on the display. Thus, the analysis provided by system 200 informs decision making by the healthcare professional and can receive information from that professional based on that decision making.

Additionally and/or alternatively, at optional step 170 of the method, the system may receive an annotation from a viewer of the displayed information. For example, the system may receive an annotation in a chart, on a graph, in a table, or via any other mechanism for annotation. A healthcare professional, after reviewing the displayed information, may enter an annotation via a user interface of the system. For example, the professional may make notes about observations of the displayed information, enter questions or queries to the patient or another healthcare professional, or enter any other information in the system.

Additionally and/or alternatively, at optional step 180 of the method, a healthcare professional and/or the system may determine that a dosage of medication is missing or necessary based on the information generated by the system. For example, the system may determine that the information generated in step 140 of the method falls inside or outside a predetermined threshold. Accordingly, the system can remind the patient to take a medication, or to supplement a medication. The system may also inform the patient of the dosage change made by the healthcare professional in step 160 of the method. As an example, the system may determine that a patient's walking speed is unusually slow (i.e., below a certain threshold) two hours after a medication dosage was supposed to be taken. The threshold can be based on previous analysis of the correlation between medication dosage, timing, and walking speed. Based on this analysis, the system can determine that a medication dosage was likely missed. The system can remind the patient to take a missed dosage.

Referring to FIG. 14 is one embodiment of the method described or otherwise envisioned herein. At 410, sensor data about an activity of daily living is collected by a sensor, such as a sensor of a wearable device. According to an embodiment, the sensor data is windowed by grouping into consecutive subgroups. The windowed data may be analyzed for detection quality, a quality metric (“ADL quality indicator”) can be extracted or otherwise generated, and the effort level per window may be extracted or otherwise calculated. Any of this data may be provided to another component at any point. For example, the sensor data may be provided from the wearable device to another component of the system where analysis occurs.

At 420, information about medication intake is received by the system. This can be provided by a healthcare professional, extracted from a prescription, provided from a patient's medication diary, or from any other sources. According to an embodiment, evaluation time compared to the last medication intake can be made.

Optionally, at 430 and 440 other information is received by the system. For example, at 430 the system may provide GPS or Bluetooth derived location information to supplement or otherwise facilitate the sensor data. At 440, the system may provide data that enables an analysis of detection quality. This other information can be provided to the system along with the sensor data 410, or separate from the sensor data.

At 450, the system selects events from the data based on a context as described or otherwise envisioned herein. For example, the system may select events such as high effort levels. Many other event selection criteria are possible as described or otherwise envisioned herein. As just examples, events may be selected based on detection quality and/or time before or from last medication intake.

At 460, the system groups events by effort level. This minimizes the influence of specific activities—such as variability in the sensor data due to a freely-chosen effort level, for example relaxed walk vs. brisk walk—on the variability due to acute changes in maximum capacity resulting from medication, such as a decrease in maximum/comfortable walk speed after levodopa-based medication intake, as just one example.

At 470, the system aggregates or pools data from a certain time period. According to an embodiment data from the generated quality metric, selected for effort level, can be aggregated over a certain time period, typically on the order of days, weeks, and months. Groupings of selected events can also be aggregated based on their detection quality. Further, based on the groupings, statistical measures can be extracted from the group of events. For example, a maximum and mean can be extracted from the aggregation.

At 480, the system may smooth or otherwise post-processes the data before it is displayed. As just one example, the data may undergo non-parametric smoothing. Many other examples of post-processing data are possible.

At 490, the system displays the aggregated data for the time period. Any of the information provided to and/or generated by the system can be displayed according to at least the embodiments described or otherwise envisioned herein. As an example, the display may comprise a trend in the data showing effort level over time. Many other displays are possible.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

1. A method for analyzing medication dosage for a patient, comprising:
receiving a signal indicative of an activity behavior of the patient over a first time period, wherein the signal is collected by a sensor of a wearable device worn by the patient;
receiving information about medication intake by the patient during the first time period, wherein the information comprises both medication dosage and medication intake times during the first time period;
analyzing the received signal and received medication intake information to determine an effectiveness of the medication on the patient, wherein the effectiveness can indicate an overdosage or underdosage of the medication during the first time period;
wherein analyzing the received signal and received medication intake information comprises generating a medication status score, the medication status score configured to minimize patient-controlled variability in the activity behavior of the patient.

2. The method of claim 1, wherein the medication status score is generated by the steps of:
identifying one or more events in the received signal based on selection criteria;
extracting a quality metric from each of the identified one or more events; and
aggregating the extracted quality metrics from the one or more events for the first time period;

3. The method of claim 1, wherein the received signal and received medication intake information are received remotely from the patient.

4. The method of claim 1, wherein the sensor is an accelerometer, a gyroscope, a magnetometer, a photopletysmograph, galvanic skin response, an air pressure sensor, a thermometer, a SpO₂ sensor, an ECG sensor, or a combination thereof.

5. The method of claim 2, wherein the quality metric comprises comprises walking intensity, heart rate variability, chair rise peak power during a sit-to-stand transition, or a combination thereof.

6. The method of claim 1, wherein the medication status score is displayed on a user interface.

7. The method of claim 6, wherein the display comprises a time series visualization for some or all of the first time period.

8. The method of claim 1, further comprising the step of receiving an adjustment, in response to an indication of an overdosage or underdosage of the medication during a subset of the first time period, of a dosage of the medication.

9. The method of claim 1, further comprising the step of receiving, from a physician monitoring the patient, an annotation for a time point during the first time period.

10. A system for analyzing medication dosage for a patient, comprising:
a communication module configured to receive: (1) a signal indicative of an activity behavior of the patient over a first time period, wherein the signal is collected by a sensor of a wearable device worn by the patient; and (2) information about medication intake by the patient during the first time period, wherein the information comprises both medication dosage and intake time(s) during the first time period;
a processor configured to analyze the received signal and received medication intake information to determine an effectiveness of the medication on the patient, wherein the effect can indicate an overdosage or underdosage of the medication during a subset of the first time period, and wherein analyzing the received signal and received medication intake information comprises generating a medication status score, the medication status score configured to minimize patient-controlled variability in the activity behavior of the patient; and
a display configured to display one or more of the received signal, the medication intake information, and/or the medication status score over the first time period.

11. The system of claim 10, wherein the processor is further configured to generate the medication status score by:
identifying one or more events in the received signal based on selection criteria;
extracting a quality metric from each of the identified one or more events; and
aggregating the extracted quality metrics from the one or more events for the first time period;

12. The system of claim 10, wherein the received signal and received medication intake information are received remotely from the patient.

13. The system of claim 10, wherein the sensor is an accelerometer, a gyroscope, a magnetometer, a photopletysmograph, galvanic skin response, an air pressure sensor, a thermometer, a SpO₂ sensor, an ECG sensor, or a combination thereof.

14. The system of claim 10, wherein the processor is further configured to receive an adjustment, in response to an indication of an overdosage or underdosage of the medication during a subset of the first time period, of a dosage of the medication.

15. The system of claim 10, wherein the processor is further configured to receive, from a physician monitoring the patient, an annotation for a time point during the first time period.
